(19) United States
(12) Patent Application Publication
(10) Pub. No.: US 2021/0353462 A1
(43) Pub. Date: Nov. 18, 2021

(54) CONTROLLER FOR AN ELECTRICAL DRIVE UNIT OF AN OPHTHALMOSURGICAL HANDPIECE AND METHOD FOR OPERATING SAME, OPHTHALMOSURGICAL APPARATUS AND SYSTEM

(71) Applicant: Carl Zeiss Meditec AG, Jena (DE)

(72) Inventors: Hans FRITZ, Fridingen (DE); Christoph KÜBLER, Oberkochen (DE)

(73) Assignee: Carl Zeiss Meditec AG, Jena (DE)

(21) Appl. No.: 17/318,330

(22) Filed: May 12, 2021

(30) Foreign Application Priority Data
May 12, 2020 (DE) 10 2020 112 851.7

(51) Int. Cl. A61F 9/007; A61M 1/00

(52) U.S. Cl. CPC A61F 9/00763; A61M 1/774; A61F 9/00745

(57) ABSTRACT

Provided is a controller for an electrical drive unit—which drives a treatment needle—of an ophthalmosurgical handpiece, comprising a generator unit, which provides a control variable with control oscillation, wherein the control oscillation has a first oscillation component at a first control frequency, wherein the first oscillation component is settable depending on a first ratio of a mechanical deflection amplitude of the treatment needle to the electrical control variable at the first control frequency.

It is proposed that the generator unit is configured to provide the control oscillation of the control variable with at least one further oscillation component at a further control frequency, different than any other control frequency, in such a way that the further oscillation component is settable depending on a further ratio of the mechanical deflection amplitude of the treatment needle to the electrical control variable at the respective further control frequency.

Furthermore, also provided is a corresponding method, an ophthalmosurgical apparatus and an ophthalmosurgical system.

The invention relates to a controller as claimed in the preamble of claim 1. Furthermore, the invention relates to an ophthalmosurgical apparatus as claimed in the preamble of claim 7. Moreover, the invention relates to an ophthalmosurgical system as claimed in the preamble of claim 8. Finally, the invention also relates to a method as claimed in the preamble of claim 9.

Ophthalmosurgical apparatuses, in particular handpieces and controllers therefor, ophthalmosurgical systems comprising ophthalmosurgical apparatuses, and corresponding methods serve, inter alia, for treating lens clouding of an eye lens in a living organism, for example a human being or an animal. In medicine, lens clouding is referred to as a cataract or gray star, inter alia. One possibility for treating lens clouding provides for replacing the natural lens of the eye by an artificial lens. One technique for treating lens clouding by replacing the natural lens by an artificial lens is phacoemulsification.

One important assembly for carrying out phacoemulsification is the ophthalmosurgical handpiece, which hereinafter is referred to just as handpiece. The handpiece comprises a treatment needle, which is mechanically connected to a drive unit of the handpiece, such that the treatment needle can be driven in oscillating fashion by the drive unit during operation as intended.

An ophthalmosurgical handpiece, a controller and a control method are disclosed for example by US 2017/0134369 A1 and also by U.S. Pat. No. 10,231,870 B2. Furthermore, it is known from U.S. Pat. No. 10,231,870 B2 to act on the treatment needle in a longitudinal direction by means of the drive unit, which is in turn acted on by a sawtooth AC voltage.

It is known that heat can be generated on account of the mechanical ultrasonic movement of a treatment needle of a handpiece owing to friction during the operation on the eye. As a result, it is possible that firstly the efficiency with regard to breaking up the natural eye lens will decrease and secondly a large amount of heat is input locally into the eye, which can lead to irreversible damage to the eye.

The invention is therefore based on the object of improving a controller of the generic type, an ophthalmosurgical apparatus of the generic type, an ophthalmosurgical system of the generic type and a method of the generic type to the effect that the treatment needle can be controlled more accurately, such that in particular the abovementioned problems with regard to heat input can be better reduced.

As a solution the invention proposes a controller, an ophthalmosurgical apparatus, an ophthalmosurgical system and a method as claimed in the independent claims.

Advantageous developments emerge from features of the dependent claims.

With regard to a controller of the generic type, the invention in accordance with a first aspect proposes in particular that the generator unit is furthermore configured to provide the control oscillation of the control variable with at least one further oscillation component at a further control frequency, different than any other control frequency, in such a way that the further oscillation component is settable depending on a further ratio of the mechanical deflection amplitude of the treatment needle to the electrical control variable at the respective further control frequency.

With regard to an ophthalmosurgical apparatus of the generic type, the invention in accordance with a second aspect proposes in particular that the generator unit is furthermore configured to provide the control oscillation of the control variable with at least one further oscillation component at a further control frequency, different than any other control frequency, in such a way that the further oscillation component is settable depending on a further ratio of the mechanical deflection amplitude of the treatment needle to the electrical control variable at the respective further control frequency.

With regard to an ophthalmosurgical system of the generic type, the invention in accordance with a third aspect proposes in particular that the ophthalmosurgical apparatus is configured in accordance with the invention.

With regard to a method of the generic type, the invention in accordance with a fourth aspect proposes in particular that the control oscillation of the control variable is provided with at least one further oscillation component at a further control frequency, different than any other control frequency, wherein the further oscillation component is set depending on a further ratio of the mechanical deflection amplitude of the treatment needle to the electrical control variable at the respective further control frequency.

The invention is based on the insight, inter alia, that the ratio between the mechanical deflection amplitude of the treatment needle, in particular the cutting tip thereof, and the corresponding control variable is dependent on the frequency. This ratio is also called core admittance. Specifically, it has been found that the core admittance is a frequency-dependent variable. This is not taken into account in the prior art. By taking account of the fact that the control variable, which can comprise the control oscillation at two or more frequencies, is provided such that the respective oscillation components are set according to the respective frequency-specific core admittance, this makes it possible to be able to set the control oscillation of the treatment needle considerably more precisely and more accurately.

The effect of the invention can be manifested particularly clearly if the electrical control variable is intended to have a predefinable temporal curve profile in order to enable a specific movement of the treatment needle, in particular the cutting tip thereof. In one embodiment, the temporal curve profile can be attained by superposing oscillation components at different frequencies, for example different phase angles and/or different amplitudes. If only the core admittance at one frequency or control frequency is taken into account in such a case, this can have the consequence that the mechanical oscillation actually effected by the treatment needle, in particular the cutting tip thereof, deviates significantly from the desired movement. This problem can be eliminated, but at least reduced, by the invention. Specifically, when providing the electrical control variable, the invention takes account of the frequency-specific core admittances which are different for the respectively different control frequencies. Preferably, the frequency-specific core admittance is taken into account for each oscillation component. The deviation of the temporal curve profile of the electrical control variable is therefore dependent, inter alia, on the respective frequency-specific core admittances. Overall, the invention makes it possible not only to improve the efficiency when breaking up the natural eye lens, but moreover also to reduce the heat input into the eye. Overall, the treatment of the eye can thus be improved.

The controller can be configured as a separately handlable unit, which can have a dedicated housing. The controller can be configured as stationary or else mobile, in particular as a portable unit. If the controller has a dedicated housing, for example, its units or assemblies can at least in part be concomitantly accommodated by the housing. The controller can furthermore also comprise an operating device or be configured for connection to such an operating device. By means of the operating device, the controller or the handpiece connected thereto can be operated in a predefined manner. Furthermore, the controller can also comprise an output interface, to which a display device can be connected, in order to be able to display one or more operating states of the controller or else the settings thereof.

The drive unit can be configured as an electrostatic drive unit or else as an electromagnetic drive unit. As an electrostatic drive unit, the drive unit can be configured in a piezo-based fashion. For this purpose, the drive unit can comprise one or else a plurality of piezoelectric elements. The piezo-based drive unit formed as a result makes it possible to utilize the effect that the piezoelectric elements, to which for example an electrical voltage is applied as electrical control variable, alter their mechanical dimensions, for example a length or the like. The treatment needle is mechanically connected to the piezo-based drive unit, such that the desired drive effect can be achieved during operation as intended. In the case of an electromagnetic drive unit, by contrast, an electromagnetic-mechanical transducer can accordingly be provided, which can be acted on for example by means of an AC current as electrical control variable. Using a magnetic field, for example, a magnetizable actuator of the transducer can be actuated in order to be able to produce the desired mechanical movement. The electrical drive unit has electrical terminals at which the electrical control variable can be applied to it in order that the desired mechanical movement for the treatment needle can be provided.

The generator unit is preferably an electronic unit configured to provide the electrical control variable for the electrical drive unit. For this purpose, the generator unit can comprise an electronic circuit, for example an inverter or the like. Furthermore, the generator unit can, of course, also comprise a program-controlled computer unit, provided in addition to the hardware circuit or else as an alternative thereof. The generator unit thus supplies the electrical control variable. The generator unit is configured to provide the control oscillation of the control variable with a first oscillation component at a first control frequency. The first control frequency is preferably in an ultrasonic range, wherein the first control frequency is preferably greater than approximately 10 kHz, and is particularly preferably in a range of approximately 40 kHz. The first control frequency can be for example approximately at a resonant frequency of the treatment needle. The first oscillation component, for example a first amplitude, can be set by means of the generator unit depending on a first ratio of a mechanical deflection amplitude of the treatment needle to the electrical control variable at the first control frequency. This core admittance may have been ascertained separately before operation as intended commenced.

In order to provide the first oscillation component, the generator unit can comprise at least one suitable electronic oscillator, which is preferably settable in order to be able to set the first control frequency in a predefined manner. The signal provided by a first oscillator can then be fed to a first amplifier, the gain of which can likewise be set. In order to be able to set the gain factor, the generator unit can comprise an amplitude factor unit, which, on the basis of the first control frequency, ascertains the assigned first core admittance or the assigned first ratio of the mechanical deflection amplitude of the treatment needle to the electrical control variable and correspondingly sets the first amplifier. The setting of the first amplifier can comprise the setting of its gain factor.

Furthermore, the generator unit is configured to provide the electrical control variable additionally with at least one second oscillation component at a second control frequency. For this purpose, the generator unit can comprise a separate second oscillator, which is preferably likewise settable, such that the desired second control frequency can be set by means of the second oscillator. The signal supplied by the second oscillator can then be fed to a second amplifier, which—in a similar manner to the first amplifier—is configured such that it is settable with regard to its gain factor. By means of the amplitude factor unit, the second control frequency can be ascertained and—just like for the first control frequency—the gain factor of the second amplifier can be set depending on a second core admittance or the mechanical deflection amplitude of the treatment needle with respect to the electrical control variable at the second control frequency. This can be correspondingly continued for further oscillation components at further control frequencies.

Therefore, the electrical control variable does not just comprise a single oscillation component at the first control frequency, but rather it also comprises at least one second oscillation component at a second control frequency. Preferably, the first frequency is the lowest frequency. In this case, the first frequency can also be referred to as the fundamental frequency. Particularly advantageously, the further control frequencies can be harmonics of the first control frequency. Preferably, for each of the further oscillation components, provision is made for separate setting depending on the respective individual frequency-specific further core admittances at the respective further control frequencies. Particularly advantageously, the electrical control variable is provided as a single signal. A respective oscillation component preferably comprises oscillation in accordance with a sinusoidal shape.

The invention thus affords the possibility of setting the electrical control variable in such a way that an oscillation behavior of the treatment needle, in particular of the cutting tip thereof, can be set very specifically in a wide setting range. For this purpose, the corresponding ratios or core admittances can be ascertained separately for the respective control frequencies for example for a respective individual handpiece or else for a respective assembly of handpieces. The setting can be carried out with the aid of a frequency spectrum analyzer.

In order to be able to attain a specific predefined type of oscillation of the treatment needle, the electrical control variable can provide a plurality of oscillation components of corresponding amplitude and optionally also taking account of respective phase angles with respect to one another. Taking account of the respective frequency-related core admittances, the desired mechanical oscillation of the treatment needle can then be set in virtually any manner desired and with virtually any accuracy desired. This allows the problems present in the prior art to be reduced or even totally avoided.

In accordance with one development, it is proposed that the controller comprises a storage unit, in which are stored, at least in the manner assigned to the respective control frequencies, individual values for the respective ratios of the mechanical deflection amplitude of the treatment needle to the electrical control variable. The storage unit allows these values to be provided such that they are retrievable during operation as intended. The storage unit can be comprised at least partly by the controller. Furthermore, there is also the possibility, of course, that the storage unit is configured at least partly separately from the controller and/or is arranged in the latter or is in communication connection via a communication connection to the controller. By way of example, the storage unit can also be comprised at least partly by a database, preferably provided by the manufacturer. This allows the individual values for the ratios of the mechanical deflection amplitude of the treatment needle to a electrical control variable not only to be provided centrally, but also to be maintained and updated centrally, and optionally also subsequently allows new individual values to be able to be provided for control frequencies that have not yet been stored previously. As a result, the functionality can be considerably improved. The storage unit can be configured as an electronic storage unit, for example in the manner of a read only memory (ROM), a random access memory (RAM), in the manner of a hard disk, in the manner of a USB stick, combinations thereof and/or the like. One advantageous configuration can furthermore provide for the storage unit to be arranged at least partly in the ophthalmosurgical handpiece. This has the advantage that the individual values can be ascertained for a respective individual ophthalmosurgical handpiece and can be stored in an assignable manner therein. As a result, it is possible to produce the controller independently of respectively required individual values for the ratios of the mechanical deflection amplitude of the treatment needle to the electrical control variable and to correspondingly adapt it individually to a respective ophthalmosurgical handpiece. Specifically, it can be provided that as a result of the handpiece being connected to the controller, the storage unit arranged therein is coupled to the controller in terms of communication technology and the values ascertained for this specific ophthalmosurgical handpiece for the ratios of the mechanical deflection amplitude of the treatment needle to the electrical control variable are thus provided for the controller. This has the further advantage that in the event of the ophthalmosurgical handpiece being changed, at the same time the individual values for the ratios of the mechanical deflection amplitude of the treatment needle to the electrical control variable can also be adapted correspondingly in an automated manner. In this configuration, therefore, in general the values assigned to the respective specific ophthalmosurgical handpiece for the ratios of the mechanical deflection amplitude of the treatment needle to the electrical control variable are available in the controller in an automated manner.

It is furthermore proposed that the generator unit is furthermore configured to superpose a plurality of oscillation components at mutually different control frequencies in order to provide a predefinable oscillation waveform of the control variable. In principle, the superposing can be realized by an addition function, for example. For this purpose, provision can be made for superposing predefined suitable oscillation components with respective control frequencies in order to realize the predefinable oscillation waveform. Supplementarily or alternatively, however, the superposing can also comprise maximum value formation, absolute value formation and/or other combination possibilities. The oscillation components to be superposed are preferably sinusoidal oscillations. In alternative configurations, however, it can also be provided that not only sinusoidal oscillations but also other oscillation waveforms can be used for the superposing, for example rectangular oscillations, triangular oscillations or the like, in order to be able to attain a desired oscillation waveform of the control variable.

Furthermore, it is proposed that the generator unit is furthermore configured to carry out the superposing at least partly as modulation. For this purpose, the controller, in particular the generator unit, can comprise a modulator that can realize the desired modulation. For example, an amplitude modulation, a frequency modulation, a phase modulation, a quadrature modulation, combinations thereof and/or the like can be provided as modulation method. The modulation can be realized using a corresponding mathematical function.

Preferably, the generator unit provides the control variable in such a way that a first time period assignable to an outgoing movement of the treatment needle is shorter than a second time period assignable to a return movement of the treatment needle. The outgoing movement of the treatment needle means a movement to a maximum positive deflection of the treatment needle proceeding from a, preferably central, rest position. In the case of a longitudinal movement, the cutting tip of the treatment needle thus moves away from a housing of the handpiece, in particular from the drive unit, during the outgoing movement. The return movement is then inverse, that is to say that the cutting tip of the treatment needle moves toward the housing, in particular the drive unit, of the handpiece. In a dual manner, the outgoing movement and the return movement can also be defined for a torsional oscillation of the treatment needle. Particularly in the case of longitudinal oscillation of the treatment needle, what can be achieved thereby is that the outgoing movement of the treatment needle is effected in a short time period, as a result of which it is possible to achieve a good effect with regard to the emulsification of the eye lens to be removed. At the same time, the return movement in a longer time period makes it possible to reduce a heat effect in the operation site on the eye. Preferably, the control variable is provided in such a way that a continuous transition from the outgoing movement of the cutting tip of the treatment needle to the return movement of the cutting tip of the treatment needle and/or vice versa can be achieved.

In accordance with one advantageous development, it is proposed that the controller comprises a sensor unit for detecting a movement of the treatment needle, wherein the sensor unit is configured to output a sensor signal depending on the detected movement of the treatment needle, and wherein the generator unit is furthermore configured to analyze the sensor signal spectrally with regard to contained frequencies taking account of the ratios of the mechanical deflection amplitude of the treatment needle to the electrical control variable at the respective control frequencies and to determine the control variable depending on the analyzing. The sensor unit can be configured as a separate unit connected to the controller, in particular to an evaluation unit of the controller, in terms of communication technology. The evaluation unit in turn can be coupled to the generator unit or a generator control unit of the controller in terms of communication technology, in order to be able to influence the provision of the control variable. The generator control unit serves, inter alia, to provide one or more control signals for controlling the generator unit. Furthermore, the generator control unit, in particular the evaluation unit, is preferably configured to be able to access the storage unit, such that the frequency-specific individual values stored there for the ratios of the mechanical deflection amplitude of the treatment needle to the electrical control variable can be made available for the evaluation. Furthermore, the sensor unit can be configured to be arranged with the handpiece, in particular in the region of the treatment needle and/or the drive unit of the handpiece, in order to be able to detect the mechanical movement of the treatment needle, in particular of the cutting tip. The sensor unit can furthermore also be at least partly a part of the handpiece, in particular of the drive unit, which part for this purpose, preferably during operating pauses during operation as intended, is able to provide the sensor signal depending on the mechanical oscillation of the treatment needle. In the case of a piezoelectric drive unit, the sensor unit can be for example a separate piezoelement of the drive unit. Furthermore, there is also the possibility, of course, of the sensor unit using further methods in order to detect the mechanical movement of the treatment needle, for example optical detection methods, magnetic detection methods and/or the like.

The generator control unit, particularly if it comprises the evaluation unit, is configured to analyze the sensor signal spectrally with regard to frequencies contained. For this purpose, a frequency analysis unit can be provided, which is able to carry out a spectral analysis using a Fourier transformation, a Laplace transformation and/or the like. In this way, a frequency spectrum of the sensor signal can be obtained, which can then be evaluated taking account of the frequency-specific ratios of the mechanical deflection amplitude of the treatment needle to the electrical control variable. This signal can then be used by the generator control unit in order that the control variable provided by the generator unit can be better adapted to the treatment needle. In particular, it is possible to compare this signal, which can be an actual signal, with a setpoint signal, which can likewise be predefined on the basis of the individual values stored in the storage unit for the ratios of the mechanical deflection amplitude of the treatment needle to the electrical control variable at the different control frequencies. What can furthermore be achieved as a result is that it is possible to correct deviations from the setpoint signal for the control variable which have been caused for example by the generator unit, electrical lines, the handpiece and/or the like. For this purpose, a comparison unit can be provided, for example, which can be comprised by the generator control unit, in particular the evaluation unit. Both the evaluation unit and the sensor unit are preferably electronic units which can comprise an electronic hardware circuit. At least the evaluation unit can furthermore also comprise or be formed by a computer unit. The sensor signal is preferably an electrical sensor signal, for example a voltage signal, a current signal or the like. It can be an analog signal or a digital signal.

Furthermore, it is proposed that the individual values for the ratio of the mechanical deflection amplitude of the treatment needle to the electrical control variable at least for the control frequencies are determined during a calibration process. Even if the calibration process can in principle be carried out during operation as intended, nevertheless the calibration process is however preferably provided outside operation as intended. The calibration process can be carried out using the controller to which the respective handpiece is connected. This can be provided during the production of a respective handpiece of the like. As a result, functional properties of the handpiece can already be detected and documented during production. Furthermore, the calibration process can, of course, also be carried out on a calibration device specifically provided for the calibration process. By way of example, it can be provided that, for the purpose of calibration, a high-speed camera detects the movement of the treatment needle. The high-speed camera can communicate corresponding image data to the evaluation unit, which determines the mechanical movement of the treatment needle. On the basis of the mechanical movement of the treatment needle, a frequency spectrum can then be determined, as has already been explained above for the control variable. The control variable used with respect to this detected mechanical movement of the treatment needle can likewise be analyzed spectrally, such that a frequency spectrum can be provided here as well. The frequency spectra ascertained in this way can then be used to determine the values for the individual ratios. The values obtained in this way are then preferably stored in the storage unit. Alternatively, the individual values for the ratio of the mechanical deflection amplitude of the treatment needle to the electrical control variable can for example also be ascertained without a corresponding high-speed camera, by virtue of the fact that the individual values for the ratio of the mechanical deflection amplitude of the treatment needle to the electrical control variable can also be determined simply from knowledge of the corresponding amplitudes in the case of a purely harmonic oscillation. A camera which can be used to estimate the oscillation amplitudes at selectively settable frequencies sufficiently accurately is then sufficient for the calibration. Such cameras are available in a cost-effective manner. Moreover, in this way, when determining the individual values for the ratio of the mechanical deflection amplitude of the treatment needle to the electrical control variable, a Fourier transformation can be avoided, although it may then still be required at a later juncture.

The advantages and effects indicated for the controller according to the invention are, of course, equally also applicable to the ophthalmosurgical apparatus according to the invention, the system according to the invention and the method according to the invention, and vice versa. In principle, therefore, apparatus features can thus also be formulated as method features, or vice versa.

Further features of the invention are evident from the claims, the figures and the description of the figures. The features and combinations of features mentioned in the description above and the features and combinations of features mentioned in the description of the figures below and/or shown in the figures alone may be used not only in the respectively specified combination, but also in other combinations, without departing from the scope of the invention. Hence, embodiments of the invention which are not explicitly shown and explained in the Fig., but which emerge and are producible by way of separated combinations of features from the explained embodiments, should also be considered to be encompassed and disclosed. Embodiments and combinations of features which therefore do not have all the features of an originally worded independent claim should also be considered to be disclosed. Furthermore, embodiments and combinations of features which go beyond or deviate from the combinations of features set out in the dependency references of the claims should be considered to be disclosed, in particular by virtue of the embodiments set out above.

In the figures:

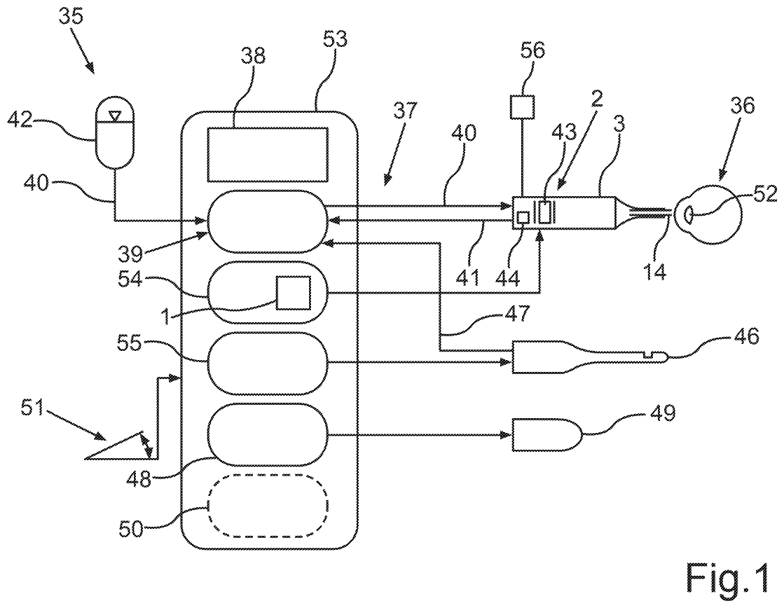

FIG. 1 shows a schematic illustration of one exemplary embodiment of an ophthalmosurgical system with one exemplary embodiment of an ophthalmosurgical apparatus comprising an ophthalmosurgical handpiece connected to a controller of the ophthalmosurgical apparatus;

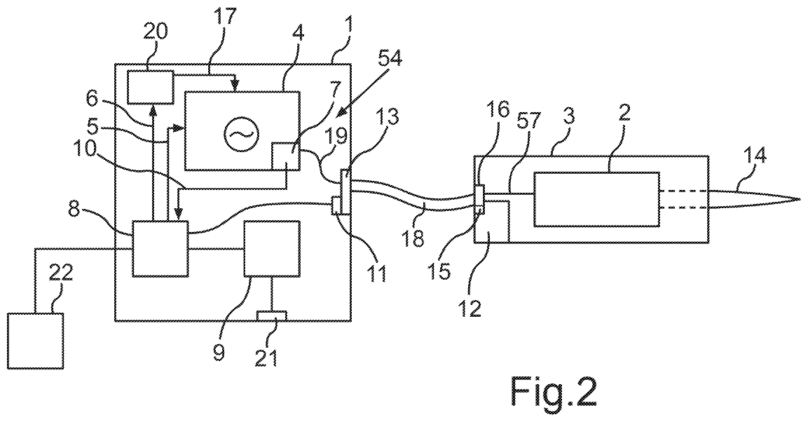

FIG. 2 shows a schematic illustration of a simplified and reduced block view of the ophthalmosurgical apparatus in accordance with FIG. 1;

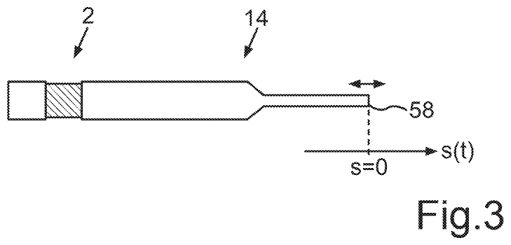

FIG. 3 shows a schematic illustration of the movement of a cutting tip of the treatment needle in accordance with FIG. 1;

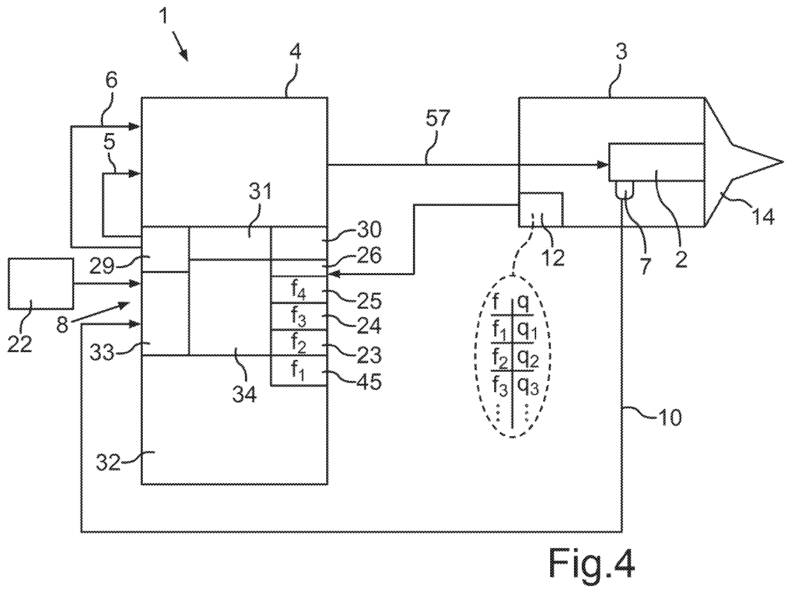

FIG. 4 shows a schematic block illustration of the controller and of the handpiece connected to the controller in accordance with FIG. 2;

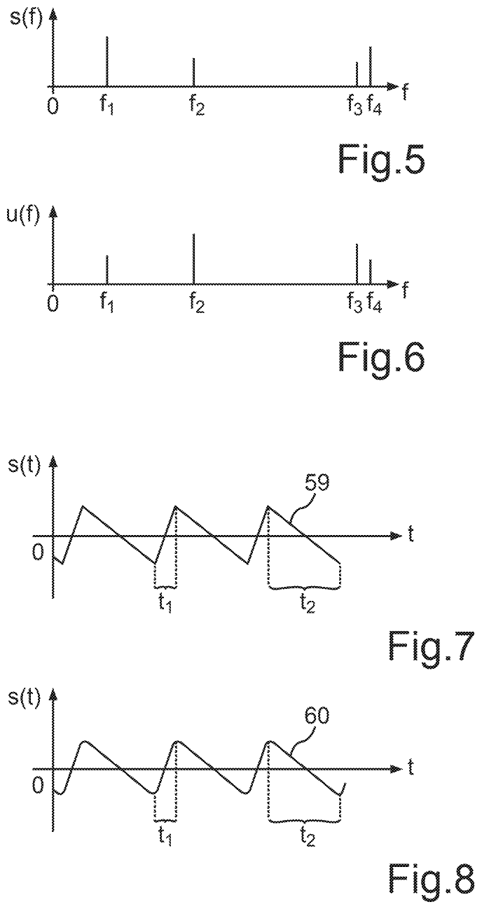

FIG. 5 shows a schematic diagram illustration of a spectral analysis of a mechanical movement of the cutting tip of the treatment needle in accordance with FIG. 3;

FIG. 6 shows a schematic diagram illustration of a spectral analysis of a control voltage for the piezoelectric drive unit in accordance with FIG. 2 in order that the cutting tip of the treatment needle effects a mechanical movement in accordance with FIG. 5;

FIG. 7 shows a schematic diagram illustration of an approximately triangular mechanical movement of the cutting tip of the treatment needle in accordance with FIG. 3; and

FIG. 8 shows a schematic diagram illustration of a mechanical movement—approximated to the triangular shape in accordance with FIG. 7—of the cutting tip of the treatment needle in accordance with FIG. 3.

In the figures, identical reference signs designate identical features and functions.

FIG. 1 shows a schematic illustration of an ophthalmic microsurgical system or an ophthalmosurgical system 35 for phaco-surgery on a human eye 36. The illustration in accordance with FIG. 1 shows some components of the system 35 symbolically for the simplified explanation of the basic general functioning of the system 35.

The system 35 comprises a device unit 53, which can be for example a control panel or the like. Preferably, an operating unit 38 is arranged in or on the device unit 53. Furthermore, a fluidic system 39 comprising a pump and a control unit for controlling the pump and connected components is preferably arranged in the device unit 53. The fluidic system 39 comprises an irrigation apparatus with an irrigation branch 40 and an aspiration apparatus with an aspiration branch 41. The irrigation apparatus comprises a container 42 for rinsing liquid, for example a BSS solution, which is a fluid for irrigation and which is guided to a phaco-handpiece. The phaco-handpiece is an ophthalmosurgical handpiece 3, referred to hereinafter just as handpiece. The aspiration apparatus is connected to the handpiece 3. The handpiece 3 comprises a drive unit 2 with piezoelectric elements 43, by means of which a hollow needle 14 as treatment needle of the handpiece 3 is excited to effect mechanical oscillation. The hollow needle 14 has a cutting tip 58 (FIG. 3), which is brought into contact with the eye lens 52 for the purpose of emulsifying said eye lens 52. In the present case, an ultrasonic unit 54 of the device unit 53 comprises at least one controller 1 having at least one AC voltage generator 4 as generator unit and having a generator control unit 8 (FIG. 2).

The device unit 53 furthermore comprises a control unit 55. The control unit 55 can also be configured for controlling a vitrectomy handpiece 46, which, in particular, can be a constituent part of the ophthalmosurgical system 35. Preferably, the vitrectomy handpiece 46 is also connected to the fluidic system 39, in particular by an aspiration line 47. Moreover, provision can be made for a further instrument control unit 48, the latter controlling a preferably available further surgical instrument 49, for example for diathermy. Moreover, the system 35 and, more particularly, the device unit 53 can comprise further modules and control units and systems, which are represented symbolically by the unit 50. Moreover, the ophthalmosurgical system 35 preferably comprises a foot control panel 51, which is connected to the device unit 53. Functions of the ophthalmosurgical system 35 can be set by means of the foot control panel 51. Moreover, FIG. 1 schematically shows a natural eye lens 52 in a human eye 36.

In an alternative embodiment, provision can be made for the ophthalmosurgical system 35 to comprise a tank 44 (FIG. 1) that is separate from the container 42. In a further embodiment, provision can be made for the separate tank 44 to be arranged in the handpiece 3. Provision can also be made for a first separate tank 44 to be arranged in the handpiece 3 and for a further separate tank 56 to be arranged externally to the handpiece 3. This further separate tank 56 external to the handpiece 3 can be connected in fluid-guiding fashion to the first separate tank 44 arranged in the handpiece 3.

FIG. 2 shows, in a schematic block view, a reduced illustration of an ophthalmosurgical apparatus 37 of the ophthalmosurgical system 35 in accordance with FIG. 1. The ophthalmosurgical apparatus 37 comprises the handpiece 3. The piezo-based drive unit 2 serves as a mechanical drive for the treatment needle 14 of the handpiece 3. In alternative configurations, a magnetically based drive unit can also be provided instead of or in addition to the piezo-based drive unit 2. The drive unit 2 is configured to excite the treatment needle 14 to mechanical oscillations in such a way as to generate a mechanical oscillation in a frequency range of approximately 20 kHz to approximately 80 kHz, preferably in a range at approximately 40 kHz. In this case, an oscillation amplitude can be approximately 100 μm.

The handpiece 3 furthermore comprises an EEPROM 12 as data memory for storing handpiece-specific data. The handpiece-specific data can comprise for example at least identification data specific to the respective handpiece 3. The ophthalmosurgical handpiece 3 furthermore comprises a communication interface 15, which is connected to the EEPROM 12 and serves for establishing a communication connection between the EEPROM 12 and the controller 1.

The ophthalmosurgical apparatus 37 furthermore comprises the controller 1, which serves to operate the handpiece 3 in a predefinable manner. The controller 1 is therefore configured for the handpiece 3, which is drivable by means of the piezo-based drive unit 2, for processing an eye lens 52. For this purpose, provision is made for the controller 1 to comprise a supply interface 13 comprising a communication interface 11. Correspondingly, the handpiece 3 also comprises a supply interface 16 comprising the communication interface 15. The supply interface 13 is releasably connected via a supply line 18 to the supply interface 16 of the handpiece 3 by way of a plug connection (not illustrated further). As a result, the controller 1 can be connected to different handpieces 3.

The controller 1 comprises the controllable AC voltage generator 4, which is connected via electrical lines 19 to the supply interface 13 and provides there an AC voltage 57 as control variable for the drive unit 2. A corresponding electrical connection is provided at the handpiece, such that the drive unit 2 can be connected to the supply lines 19. In the present case, the AC voltage has an amplitude that is settable in a range of approximately 20 V to approximately 30 V. The amplitude of the AC voltage 57 is settable depending on a control signal 6 for the amplitude. The control signal 6 is provided by the generator control unit 8. Even if the control variable is formed by an AC voltage in the present case, in alternative configurations the control variable can also be formed by some other suitable, preferably electrical, variable, for example an AC current or the like. The type of the control variable is preferably chosen in a manner adapted to the configuration of the drive unit 2.

In order to set an amplitude of the AC voltage 57, the controller 1 comprises a power supply unit 20, which provides a supply voltage 17 for the AC voltage generator 4. By means of the control signal 6 for the amplitude, it is possible to set a value of the supply voltage 17 and consequently also the amplitude of the AC voltage provided by the AC voltage generator 4. Control oscillation of the AC voltage 57 provided by the AC voltage generator 4 can be set by means of a further control signal 5.

In order to be able to ascertain the control signals 5, 6, the AC voltage generator 4 comprises a voltage sensor 7 as sensor unit, said voltage sensor being connected to the electrical lines 19. The voltage sensor 7 delivers an electrical voltage or feedback voltage 10 as sensor signal, which voltage represents an electrical operating state variable and is dependent on an operating state of the handpiece 3, in particular of the treatment needle 14. For the purpose of detecting the feedback voltage 10, the AC voltage generator 4 is momentarily deactivated in each case.

The feedback voltage 10 is fed to a generator control unit 8 of the controller 1, said generator control unit providing the control signals 5, 6 depending on the feedback voltage 10. At the same time, the AC voltage generator 4 can be controlled by means of the generator control unit 8 in such a way that it is momentarily deactivated for the purpose of detecting the feedback voltage 10. In the present case, the deactivation time period is approximately 450 μs. In the present case, this time period is repeated at a time interval of approximately 10 ms.

The generator control unit 8 furthermore comprises an evaluation unit 9 of the controller 1, said evaluation unit being configured to determine state variables or operating variables of the controller 1, in particular in regard to the feedback voltage 10 and the AC voltage 57, in order that the control signals 5, 6 can be set depending thereon. Furthermore, a display device can be connected to a display interface 21 of the controller 1, said display interface being coupled to the controller 1. A display of operating states of interest to the surgeon or user can be achieved by means of the display device. The display device is not illustrated in the figures.

The evaluation unit 9 comprises for this purpose a program-controlled computer unit, which realizes the required functions of the evaluation unit 9 and of the generator control unit 8. In addition—as required—a hardware circuit can also be provided.

Furthermore, an operating device 22 is connected to the controller 1, in particular to the generator control unit 8, and the surgeon can set the control signals 5, 6 by means of said operating device, specifically depending on current progress of an operation or a current situation of an operation.

The communication interface 11 and the communication interface 15 are coupled to one another in a wired manner via the supply line 18. As a result, a communication connection can also be established between the controller 1 and the handpiece 3. This allows, inter alia, identification data of the handpiece 3 to be read out from the EEPROM 12 therefrom, and to be made available for the generator control unit 8. It is not only for this purpose that the generator control unit 8 is connected to the communication interface 11.

There is furthermore the possibility of storing specific operating data from the generator control unit 8 for the respective handpiece 3 in the EEPROM 12 thereof. As a result, these data can be specific to a respective handpiece 3 and be used for individually setting the controller 1. In this regard, it can be provided that upon the handpiece 3 being connected to the controller 1, the corresponding data are read out from the EEPROM 12 and communicated to the generator control unit 8. The generator control unit 8 can then correspondingly control the AC voltage generator 4, such that a predefined function of the handpiece 3 can be achieved. Upon an operation being ended, provision can be made for data present in the generator control unit 8 with regard to the handpiece 3 to be stored again in the EEPROM 12 thereof for a later use. As a result, it is possible to improve the operation of the handpiece 3 at a respective controller 1.

FIG. 3 shows, in a schematic illustration, how the cutting tip 58 of the treatment needle 14 moves mechanically during operation as intended. The movement shown in FIG. 3 is a movement of the cutting tip 58, specifically in a longitudinal direction or in the direction of a longitudinal extent of the treatment needle 14. However, the following explanations are equally applicable to a torsional movement or rotational movement, too. During operation as intended, the treatment needle 14 and in particular the cutting tip 58 thereof are driven in a longitudinal direction by the drive unit 2 in the present case. The cutting tip 58 thus carries out a to and fro movement, wherein the outgoing movement of the cutting tip 58 is a movement during which the cutting tip 58 moves away from the drive unit, whereas the opposite movement, the movement back or the return movement, is a movement during which the cutting tip 58 moves toward the drive unit 2. In a path diagram illustrated in an assigned manner in FIG. 3, a position s=0 is illustrated schematically, which position denotes a rest position of the cutting tip 58 when the drive unit 2 is not driving the treatment needle 14 outside operation as intended.

FIG. 4 shows the respective function blocks in a further schematic block illustration of the controller 1 and of the handpiece 3 connected to the controller 1. In order to explain the details, it is assumed in this embodiment, for example, that the AC voltage 57 encompasses the oscillation of four control frequencies, specifically the frequencies f₁ to f₄ as also explained below with reference to FIGS. 6 and 7.

It is evident from FIG. 4 that the handpiece 3 comprises not only the treatment needle 14 and the piezoelectric drive unit 2, but also the storage unit 12, configured as an EEPROM in the present case. In the storage unit 12, there are stored for example individual values for core admittances q₁ to q₄ in an assigned manner for respective control frequencies f₁ to f₄, for example in the manner of a look-up table. In this case, the core admittance q₁ is assigned to the frequency f₁, the core admittance q₂ is assigned to the frequency f₂, and the core admittance q₃ is assigned to the frequency f₃. The same correspondingly applies to f₄, q₄. As necessary, the table thereby formed can be correspondingly continued for further control frequencies. These individual values in the storage unit 12 are ascertained specifically for the handpiece 3. These values can preferably be ascertained during a calibration process outside intended operation of the handpiece 3, preferably during the production of the handpiece 3.

For this purpose, the mechanical movement of the treatment needle 14, in particular of the cutting tip 58 thereof, can be recorded by a suitable high-speed camera and can be analyzed with regard to mechanical and temporal variables by means of an evaluation unit connected to the camera. The movement sequence ascertained as a result can then be analyzed spectrally, for example by performing a Fourier transformation, in particular a discrete Fourier transformation, by means of a program-controlled computer unit suitable for this purpose. A corresponding Fourier transformation can likewise be carried out for the AC voltage 57 as control variable. It is thereby possible to ascertain the corresponding ratios or core admittances q in a frequency-specific manner and to correspondingly store them in the storage unit 12. This is not illustrated in the figures, however. Alternatively, it is possible to obviate the Fourier transformation in connection with the determination of the corresponding ratios or core admittances q if the calibration is carried out separately for the individual harmonic oscillations. A corresponding Fourier transformation is then necessary, however, at a later juncture, that is to say during the analysis of a detected movement pattern into its individual oscillation components.

The controller 1 comprises oscillators 23, 24, 25, 45 in the present configuration, which oscillators are configured such that they are settable in this configuration. The oscillators 23, 24, 25, 45 are formed by electronic hardware circuits in the present case. In alternative configurations, however, they can at least in part also be formed or comprised by a program-controlled computer unit. The oscillators 23, 24, 25, 45 are part of the generator control unit 8 in the present case. Each of the oscillators 23, 24, 25, 45 delivers an oscillator signal, which, in the present case, is an AC voltage signal at a control frequency set differently from one another in each case, said signal having a predefined amplitude.

The generator control unit 8 furthermore comprises an oscillator control facility 26 configured to control the oscillators 23, 24, 25, 45 with regard to the frequency to be set and to set the oscillators 23 to 25 and 45 accordingly with regard to the frequencies to be set. Furthermore, the oscillator control facility 26—as is evident from FIG. 4—is coupled to the storage unit 12 of the handpiece 3 in terms of communication technology. A core admittance ascertaining unit 30 of the generator control unit 8 is likewise coupled to the storage unit 12 of the handpiece 3 in terms of communication technology. In the present case, the core admittance ascertaining unit 30 ascertains the respectively assigned core admittances q₁ to q₄ for the frequencies f₁ to f₄. The core admittance ascertaining unit 30 makes these values available for an amplitude factor unit 34, likewise comprised by the generator control unit 8. The amplitude factor unit 34 receives from the oscillators 23 to 25 and 45 the corresponding AC voltage signals with respectively normalized amplitudes. The amplitude factor unit 34 receives from the core admittance ascertaining unit 30 the corresponding frequency-specific core admittances q₁ to q₄. The amplitude factor unit 34 ascertains for the respective frequencies f₁ to f₄, taking account of the core admittances q₁ to q₄, respective amplitude values for the oscillation at the respective frequencies f₁ to f₄. These amplitude values are used for ascertaining respective frequency-specific individual AC voltages. The individual AC voltages are then fed to a superposing unit 31 of the generator control unit 8, which superposes the individual AC voltages to form a common AC voltage. This total AC voltage is fed to a control signal unit 29, which generates the corresponding control signals 5, 6 and provides them to the AC voltage generator 4 for generating the AC voltage 57.

In the present configuration, provision is furthermore made for the voltage sensor 7 to output the feedback voltage 10 in a predefined manner—as explained above—, said feedback voltage likewise being fed to the generator control unit 8. In the present case, the feedback voltage 10 is fed to a frequency analyzer 33, which analyzes the feedback voltage 10 of the voltage sensor 7 with regard to its frequency components. A Fourier transformation or discrete Fourier transformation can likewise be provided for this purpose. The frequency analyzer 33 is furthermore configured to carry out a comparison function. The feedback voltage 10 analyzed with regard to the frequency spectrum can be analyzed further taking account of the core admittances q₁ to q₄ provided by the core admittance ascertaining unit 30. An actual signal can be provided as a result. Said actual signal can be compared with a predefined setpoint signal in order to determine a difference control signal. The latter can then be taken into account for determining the control variable. It is thereby possible to achieve closed-loop control with regard to the AC voltage 57, such that undesired influencing effects, for example resulting from the AC voltage generator 4 or the like, can be reduced.

The generator control unit 8 furthermore comprises a waveform generator 32, which can be used to set or predefine a temporal waveform for the mechanical movement of the treatment needle 14, in particular of the cutting tip 58. The waveform generator 32 makes it possible to predefine the temporal waveform of the mechanical to and fro movement of the treatment needle 14 or of the cutting tip 58. The waveform can be for example a triangular waveform, a sawtooth waveform, a sinusoidal oscillation and/or the like. The waveform generator can be correspondingly set by means of the operating device 22. Here, too, the frequency analyzer 33 can be used to ascertain the control signals 5, 6. The frequency analyzer 33, using its comparison function and taking account of the core admittances q₁ to q₄ for the frequencies f₁ to f₄, can ascertain whether the feedback voltage 10 contains the desired components to the desired extent.

It is thereby possible, with the waveform generator, to provide a setpoint signal and to be able to track as accurately as possible the waveform predefined by the waveform generator 32 for the mechanical movement of the treatment needle 14 or of the cutting tip 58, because the feedback voltage 10 can deliver a corresponding value depending on an actually performed mechanical movement of the treatment needle 14 or of the cutting tip 58.

With reference to FIGS. 5 and 6 it is evident how the invention can take effect. FIG. 5 shows, in a normalized schematic diagram, a spectral analysis of the mechanical movement of the cutting tip 58 for predefined operation as intended in a further exemplary embodiment. The mechanical movement illustrated in FIG. 5 contains the oscillation of four frequencies in the present case, specifically the frequencies f₁, f₂, f₃ and f₄. The diagram in accordance with FIG. 5 illustrates for these frequencies the corresponding proportions of the amplitudes s relative to one another.

FIG. 6 shows, in a schematic diagram like FIG. 5, a frequency spectrum for an AC voltage u as control variable that produces the mechanical movement of the cutting tip 58 in accordance with FIG. 5. It can be discerned that the AC voltage 57 here comprises the same frequency components f₁ to f₄. It is evident, however, that the frequency spectrum in accordance with FIG. 6 is required for generating the frequency spectrum in accordance with FIG. 5. The comparison between the diagrams in accordance with FIG. 5 and FIG. 6 reveals that the relative oscillation components at the respective control frequencies differ from one another. This is owing to the fact that the correspondingly assigned core admittances require corresponding setting of the relative amplitude values of the AC voltage 57 in order that the frequency spectrum in accordance with FIG. 5 can be attained. It is evident that the core admittance is taken into account in a frequency-specific manner, which explains why for example the relative amplitude component at the control frequency f₁ in the diagram in accordance with FIG. 5 is significantly greater than in the diagram in accordance with FIG. 6. This is already reversed for the control frequency f₂. This is a consequence of the deviating core admittance at the control frequency f₂ vis-à-vis the core admittance at the control frequency f₁. The same also applies to the control frequencies f₃ and f₄ as well. This explains why the invention makes it possible to control the movement of the cutting tip 58 or of the treatment needle 14 significantly better than is possible with the prior art.

FIG. 7 shows, in a schematic path-time diagram, a mechanical movement sequence of the treatment needle 14, in particular of the cutting tip 58 thereof, with a graph 59. It is evident from FIG. 7 that the cutting tip 58 performs an approximately triangular to and fro movement. It is evident from FIG. 7 that the outgoing movement is effected during a time period t₁. By contrast, the return movement is effected in a time period t₂. It is evident from FIG. 7 that the time period t₁ is considerably shorter than the time period t₂. It has been found that in particular in the case of longitudinal movements, a fast forward movement combined with a slow backward movement can significantly reduce the heat input. This movement therefore proves to be particularly advantageous in this regard.

However, the graph 59 represents an idealized form of the movement, such as can hardly be realized in practice. FIG. 8 shows, in a schematic path-time diagram like FIG. 7, an approximated movement with a graph 60, the latter making it possible to achieve a functionality comparable to that achieved with the movement in accordance with the graph 59 in FIG. 7. It can be discerned that the graph 60 shows a substantially mathematically continuous movement in contrast to the graph 59. A path-time function of this movement is preferably mathematically continuously differentiable or smooth. This movement can be achieved by modulation, for example. The modulation can be effected for example in accordance with the following formula:

s(t)=s₀*cos(ω*t+a*cos(ω*t))

s₀ denotes an amplitude of the mechanical deflection of the treatment needle and can assume a value of up to approximately 100 μm, for example. a is a weight and can determine a difference between the fast forward movement and the slow backward movement. A typical value for a can be in a range of approximately 0.1 to approximately 0.75, for example. It can preferably be approximately 0.5. ω includes the frequency in a known manner. This temporal profile can be provided by means of the waveform generator 32. By way of the processing of this signal by means of the generator control unit 8 by means of frequency analysis taking account of the frequency-specific core admittances, it is possible to set the AC voltage generator 4 for providing a corresponding AC voltage 57, such that the desired temporal profile of the oscillation of the treatment needle 14 can be attained.

Even if the invention has been explained on the basis of a longitudinal movement, nevertheless it is, of course, equally also applicable to torsional movements, in particular rotational oscillations, and combinations of torsional movements and longitudinal movements. The invention is therefore not restricted to being used exclusively in the case of longitudinal movements.

The exemplary embodiments serve exclusively for explaining the invention and are not intended to restrict the invention.

1. A controller for an electrical drive unit—which drives a treatment needle—of an ophthalmosurgical handpiece for processing an eye lens, comprising a generator unit for providing an electrical control variable for the electrical drive unit, wherein the generator unit is configured to provide the control variable with control oscillation, wherein the control oscillation has a first oscillation component at a first control frequency, wherein the first oscillation component is settable depending on a first ratio of a mechanical deflection amplitude of the treatment needle to the electrical control variable at the first control frequency, wherein the generator unit is furthermore configured to provide the control oscillation of the control variable with at least one further oscillation component at a further control frequency, different than any other control frequency, in such a way that the further oscillation component is settable depending on a further ratio of the mechanical deflection amplitude of the treatment needle to the electrical control variable at the respective further control frequency.

2. The controller as claimed in claim 1, wherein a storage unit, in which are stored, at least in the manner assigned to the respective control frequencies, individual values for the respective ratios of the mechanical deflection amplitude of the treatment needle to the electrical control variable.

3. The controller as claimed in claim 1, wherein the generator unit is furthermore configured to superpose at least a plurality of oscillation components at mutually different control frequencies in order to provide a predefinable oscillation waveform of the control variable.

4. The controller as claimed in claim 3, wherein the generator unit is furthermore configured to carry out the superposing at least partly as modulation.

5. The controller as claimed in claim 1, wherein the generator unit provides the control variable in such a way that a first time period assignable to an outgoing movement of the treatment needle is shorter than a second time period assignable to a return movement of the treatment needle.

6. The controller as claimed in claim 1, wherein a sensor unit for detecting a movement of the treatment needle, wherein the sensor unit is configured to output a sensor signal depending on the detected movement of the treatment needle, and wherein the generator unit is furthermore configured to analyze the sensor signal spectrally with regard to contained frequencies taking account of the ratios of the mechanical deflection amplitude of the treatment needle to the electrical control variable at the respective control frequencies and to determine the control variable depending on the analyzing.

7. An ophthalmosurgical apparatus comprising:
an ophthalmosurgical handpiece for processing an eye lens, said handpiece being drivable by means of a drive unit; and
a controller connectable to the ophthalmosurgical handpiece at least during operation as intended, wherein the controller comprises a generator unit for providing an electrical control variable for the electrical drive unit, wherein the generator unit is configured to provide the control variable with control oscillation, wherein the control oscillation has a first oscillation component at a first control frequency, wherein the first oscillation component is settable depending on a first ratio of a mechanical deflection amplitude of the treatment needle to the electrical control variable at the first control frequency,
wherein the generator unit is furthermore configured to provide the control oscillation of the control variable with at least one further oscillation component at a further control frequency, different than any other control frequency, in such a way that the further oscillation component is settable depending on a further ratio of the mechanical deflection amplitude of the treatment needle to the electrical control variable at the respective further control frequency.

8. An ophthalmosurgical system for processing an eye lens, at least comprising:
an irrigation apparatus,
an aspiration apparatus, and
an ophthalmosurgical apparatus,
wherein the ophthalmosurgical apparatus is configured as claimed in claim 7.

9. A method for operating an electrical drive unit 2—which drives a treatment needle—of an ophthalmosurgical handpiece serving for processing an eye lens, wherein an electrical control variable is provided with a control oscillation by means of a generator unit, wherein the control oscillation has a first oscillation component at a first control frequency, wherein the first oscillation component is set depending on a first ratio of a mechanical deflection amplitude of the treatment needle to the electrical control variable at the first control frequency, wherein the control oscillation of the control variable is provided with at least one further oscillation component at a further control frequency, different than any other control frequency, wherein the further oscillation component is set depending on a further ratio of the mechanical deflection amplitude of the treatment needle to the electrical control variable at the respective further control frequency.

10. The method as claimed in claim 9, wherein the individual values for the ratio of the mechanical deflection amplitude of the treatment needle to the electrical control variable at least for the first frequency and the at least one second frequency are determined during a calibration process.
